(19) United States
(12) Patent Application Publication
(10) Pub. No.: US 2009/0171423 A1
(43) Pub. Date: Jul. 2, 2009

(54) Head Orthosis

(76) Inventor: Fukuma Wada, Fukuoka (JP)

Correspondence Address: WENDEROTH, LIND & PONACK, L.L.P., 1030 15th Street, N.W.,, Suite 400 East, Washington, DC, 20005-1503, US

(21) Appl. No.: 12/085,160

(22) PCT Filed: Mar. 17, 2006
(86) PCT No.: PCT/JP2006/305456

§ 371 Date: May 16, 2008

(51) Int. Cl. A61N 5/06

(52) U.S. Cl. 607/88

(57) ABSTRACT

This invention relates to a head orthosis that fits about the head and radiates far-infrared radiation for activating the physiological conditions of the head. The head orthosis according to the invention comprises a radiator formed of a material containing a pulverized far-infrared radiating substance in a strip shape so as to come into retaining contact with at least a part of the temporal region of the head. The head orthosis having the radiator in contact with the temporal region of the head can irradiate the far infrared rays intensively to the temporal region of the head, while preventing the far-infrared rays from dispersing over the entire head, thereby to apply far-infrared radiation intensively to the head having the nerve tissues. Furthermore, since the radiator contains homogeneously pulverized far-infrared radiating substance, the far-infrared radiating area of the far-infrared radiating substance is made wide to prevent the interference of the far-infrared radiation, consequently to increase the amount of far-infrared radiation releasing from the far-infrared radiating substance.

### TECHNICAL FIELD

This invention relates to a head orthosis that fits about the head and radiates far-infrared radiation for activating the physiological conditions of the head.

### BACKGROUND ART

In general, it has been widely recognized that far infrared rays (rays with a wavelength of 4 to 25 micrometers, which are regarded as a growing light ray and a far-infrared ray used for medical purposes) are capable of activating the physical conditions. There have been provided various devices with a radiator formed of a material containing a far-infrared radiating substance for medical, therapeutic and health-promoting purposes and other purposes of the kind. Recently, there has been a need to develop a head orthosis capable of effectively applying far-infrared rays to the head, based on announced research results such that application of the far-infrared radiation to the head can more encourage the physiologic activation compared with that to the other parts such as the trunk and foot of the body.

There has been known a conventional head orthosis capable of applying the far-infrared rays to the head (Patent Literature 1).

The conventional head orthosis is made by attaching a radiator for far-infrared radiation to a cap-shaped net that fits about the head so as to apply the far-infrared rays to the head.

The conventional head orthosis can apply the far-infrared rays nearly evenly to the whole of the head, but disadvantageously cannot irradiate the far infrared rays intensively to the temporal region of the head containing important nervous tissue. Moreover, since a far-infrared radiating substance incorporated in the conventional head orthosis can radiate only minute amounts of radiation exposure of the far-infrared rays, this head orthosis has a problem of being incapable of thoroughly achieving the physiologic activation.

[Patent Literature 1] Japanese Utility Model Registration Publication No. 3084581

### DISCLOSURE OF THE INVENTION

This invention is made in the light of the aforementioned problems and seeks to provide a head orthosis capable of irradiating an adequate amount of far infrared rays intensively to the temporal region of the head.

As one solution to the solve the conventional technical problems according to the present invention as claimed in claim 1, there is provided a head orthosis including a radiator formed of a material containing a pulverized far-infrared radiating substance in a strip shape so as to come into retaining contact with at least a part of the temporal region of the head.

According to the aforementioned solution, the head orthosis having the radiator in contact with the temporal region of the head can irradiate the far infrared rays intensively to the temporal region of the head, while preventing the far-infrared rays from dispersing over the entire head, thereby to apply far-infrared radiation intensively to the head having the nerve tissues. Furthermore, since the radiator contains homogeneously pulverized far-infrared radiating substance, the far-infrared radiating area of the far-infrared radiating substance is made wide to prevent the interference of the far-infrared radiation, consequently to increase the amount of far-infrared radiation releasing from the far-infrared radiating substance.

As another solution to the solve the conventional technical problems according to the present invention as claimed in claim 2, there is provided the head orthosis claimed in claim 1, in which the far-infrared radiating substance is silica or graphite silica pulverized with a particle diameter of 0.001 to 0.020 μm.

According to this solution, the far-infrared radiating substance can be specified concretely to enable identification of the granularity of the specified substance.

As the other solution to the solve the conventional technical problems according to the present invention as claimed in claim 3, there is provided the head orthosis claimed in claim 2, in which the radiator is formed of thermoplastic synthetic resin mixed with the far-infrared radiating substance in a ratio by weight of 3 to 10%.

According to this solution, the mixing ratio of the far-infrared radiating substance contained in the radiator relative to the material of the radiator can be specified.

As the other solution to the solve the conventional technical problems according to the present invention as claimed in claim 4, there is provided a head orthosis including a radiator formed of a material containing a pulverized far-infrared radiating substance in a strip shape, so that the radiator can come into retaining contact with at least a part of the temporal region of the head, thereby to elastically hold the radiator onto the temporal region of the head by the elasticity of the material.

This solution can bring about the same function and effect as those of the head orthosis claimed in claim 1 and further enables the radiator to be steadily secured onto the temporal region of the head.

As the other solution to the solve the conventional technical problems according to the present invention as claimed in claim 5, there is provided the head orthosis claimed in claim 4, in which the far-infrared radiating substance is silica or graphite silica pulverized with a particle diameter of 0.001 to 0.020 μm.

According to this solution, the far-infrared radiating substance can be specified concretely to enable identification of the granularity of the specified substance.

As the other solution to the solve the conventional technical problems according to the present invention as claimed in claim 6, there is provided the head orthosis claimed in claim 5, in which the radiator is formed of thermoplastic synthetic resin mixed with the far-infrared radiating substance in a ratio by weight of 3 to 10%.

According to this solution, the mixing ratio of the far-infrared radiating substance contained

As the other solution to the solve the conventional technical problems according to the present invention as claimed in claim 7, there is provided a head orthosis including a radiator formed of a material containing a pulverized far-infrared radiating substance in a strip shape, so that the radiator can come into retaining contact with at least a part of the temporal region of the head, thereby to elastically hold the radiator onto the temporal region by the elasticity of the material and be folded in its detached state from the temporal region.

This solution can bring about the same function and effect as those of the head orthosis claimed in claim 1 and further enables the radiator to be steadily secured onto the temporal region of the head.

As the other solution to the solve the conventional technical problems according to the present invention as claimed in claim 8, there is provided the head orthosis claimed in claim 7, in which the far-infrared radiating substance is silica or graphite silica pulverized with a particle diameter of 0.001 to 0.020 μm.

According to this solution, the far-infrared radiating substance can be specified concretely to enable identification of the granularity of the specified substance.

As the other solution to the solve the conventional technical problems according to the present invention as claimed in claim 9, there is provided the head orthosis claimed in claim 8, in which the radiator is formed of thermoplastic synthetic resin mixed with the far-infrared radiating substance in a ratio by weight of 3 to 10%.

According to this solution, the mixing ratio of the far-infrared radiating substance contained in the radiator relative to the material of the radiator can be specified.

As the other solution to the solve the conventional technical problems according to the present invention as claimed in claim 10, there is provided a head orthosis including a radiator formed of a material containing a pulverized far-infrared radiating substance in a strip shape, which radiator is coupled to a strip-shaped support member in contact with the temporal region of the head.

This solution can bring about the same function and effect as those of the head orthosis claimed in claim 1 and further enables the radiator to be steadily secured onto the temporal region of the head by the support member.

As the other solution to the solve the conventional technical problems according to the present invention as claimed in claim 11, there is provided the head orthosis claimed in claim 10, in which the far-infrared radiating substance is silica or graphite silica pulverized with a particle diameter of 0.001 to 0.020 μm.

According to this solution, the far-infrared radiating substance can be specified concretely to enable identification of the granularity of the specified substance. As the other solution to the solve the conventional technical problems according to the present invention as claimed in claim 12, there is provided the head orthosis claimed in claim 11, in which the radiator is formed of thermoplastic synthetic resin mixed with the far-infrared radiating substance in a ratio by weight of 3 to 10%.

According to this solution, the mixing ratio of the far-infrared radiating substance contained in the radiator relative to the material of the radiator can be specified.

As the other solution to the solve the conventional technical problems according to the present invention as claimed in claim 13, there is provided a head orthosis including a radiator formed of a material containing a pulverized far-infrared radiating substance in a strip shape, in which the radiator is detachably coupled to a strip-shaped support member coming into contact with the temporal region of the head, and the radiator and the support member are attachable and detachable with respect to each other.

This solution can bring about the same function and effect as those of the head orthosis claimed in claim 1 and further enables the head orthosis to be disassembled and stored in a compact state when not in use.

As the other solution to the solve the conventional technical problems according to the present invention as claimed in claim 14, there is provided the head orthosis claimed in claim 13, in which the far-infrared radiating substance is silica or graphite silica pulverized with a particle diameter of 0.001 to 0.020 μm.

According to this solution, the far-infrared radiating substance can be specified concretely to enable identification of the granularity of the specified substance.

As the other solution to the solve the conventional technical problems according to the present invention as claimed in claim 15, there is provided the head orthosis claimed in claim 14, in which the radiator is formed of thermoplastic synthetic resin mixed with the far-infrared radiating substance in a ratio by weight of 3 to 10%.

According to this solution, the mixing ratio of the far-infrared radiating substance contained in the radiator relative to the material of the radiator can be specified.

As the other solution to the solve the conventional technical problems according to the present invention as claimed in claim 16, there is provided a head orthosis including a radiator formed of a material containing a pulverized far-infrared radiating substance in a strip shape, in which the radiator is coupled to a strip-shaped support member coming into contact with the temporal region of the head, and the angle between the radiator and the support member are adjustable.

This solution can bring about the same function and effect as those of the head orthosis claimed in claim 1 and further makes it possible to adjust the position of the support member relative to the parietal region in accordance with the size and shape of the head of a user.

As the other solution to the solve the conventional technical problems according to the present invention as claimed in claim 17, there is provided the head orthosis claimed in claim 16, in which the far-infrared radiating substance is silica or graphite silica pulverized with a particle diameter of 0.001 to 0.020 μm.

According to this solution, the far-infrared radiating substance can be specified concretely to enable identification of the granularity of the specified substance.

As the other solution to the solve the conventional technical problems according to the present invention as claimed in claim 18, there is provided the head orthosis claimed in claim 17, in which the radiator is formed of thermoplastic synthetic resin mixed with the far-infrared radiating substance in a ratio by weight of 3 to 10%.

According to this solution, the mixing ratio of the far-infrared radiating substance contained in the radiator relative to the material of the radiator can be specified.

As the other solution to the solve the conventional technical problems according to the present invention as claimed in claim 19, there is provided a head orthosis including a radiator formed of a material containing a pulverized far-infrared radiating substance in a strip shape, in which the radiator is coupled to a strip-shaped support member coming into contact with the temporal region of the head, and the connecting position between the radiator and the support member are adjustable in the longitudinal direction of the radiator.

This solution can bring about the same function and effect as those of the head orthosis claimed in claim 1 and further makes it possible to adjust the position of the support member relative to the parietal region in accordance with the size and shape of the head of a user.

As the other solution to the solve the conventional technical problems according to the present invention as claimed in claim 20, there is provided the head orthosis claimed in claim 19, in which the far-infrared radiating substance is silica or graphite silica pulverized with a particle diameter of 0.001 to 0.020 μm.

According to this solution, the far-infrared radiating substance can be specified concretely to enable identification of the granularity of the specified substance.

As the other solution to the solve the conventional technical problems according to the present invention as claimed in claim 21, there is provided the head orthosis claimed in claim 20, in which the radiator is formed of thermoplastic synthetic resin mixed with the far-infrared radiating substance in a ratio by weight of 3 to 10%.

According to this solution, the mixing ratio of the far-infrared radiating substance contained in the radiator relative to the material of the radiator can be specified.

### BRIEF DESCRIPTION OF THE DRAWINGS

FIG. 1 is a perspective view showing a first preferred embodiment for carrying out the present invention, with an in-use view of a head orthosis according to the invention.

FIG. 2 is an enlarged perspective view of the principal part of the head orthosis in FIG. 1.

FIG. 3 is a perspective view showing the folded state of the principal part of the head orthosis in FIG. 1.

FIGS. 4(A), 4(B) and 4(C) are side views showing modes of using the head orthosis, which are different from that in FIG. 1.

FIG. 5A is a micrograph showing a blood state before using the head orthosis in the first preferred embodiment.

FIG. 5B is a micrograph showing a blood state after using the head orthosis in the first preferred embodiment.

FIG. 6A is another micrograph showing a blood state before using the head orthosis in the first preferred embodiment.

FIG. 6B is another micrograph showing a blood state after using the head orthosis in the first preferred embodiment.

FIG. 7 is a perspective view of a second preferred embodiment of the head orthosis according to the invention.

FIG. 8 is a side view of a third preferred embodiment of the head orthosis in use according to the invention.

FIG. 9 is a plan view of FIG. 8.

FIG. 10 is an enlarged longitudinal sectional view of the principal part of the head orthosis in FIG. 8.

FIG. 11 is an enlarged perspective view of the principal part of the head orthosis in FIG. 8.

FIG. 12 is a side view of a fourth preferred embodiment of the head orthosis in use according to the invention.

FIG. 13 is a plan view of a fifth preferred embodiment of the head orthosis according to the invention.

FIG. 14 is a side view of a sixth preferred embodiment of the head orthosis according to the invention.

FIG. 15 is an enlarged perspective view of the principal part of the head orthosis in FIG. 14.

### BEST MODE FOR CARRYING OUT THE INVENTION

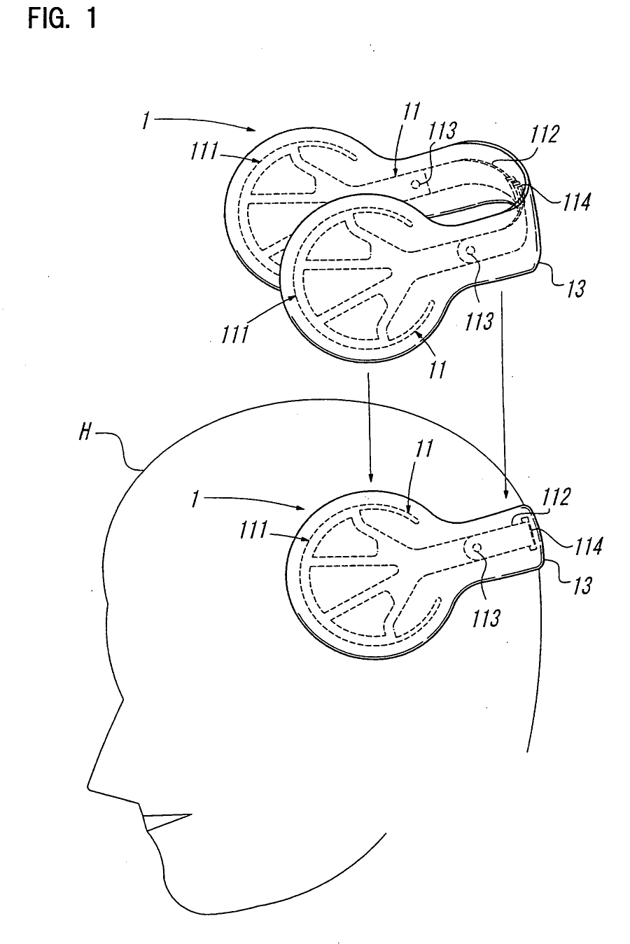

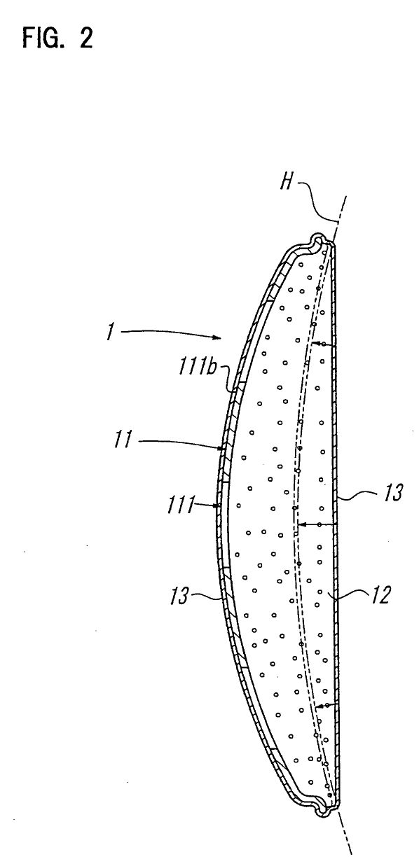

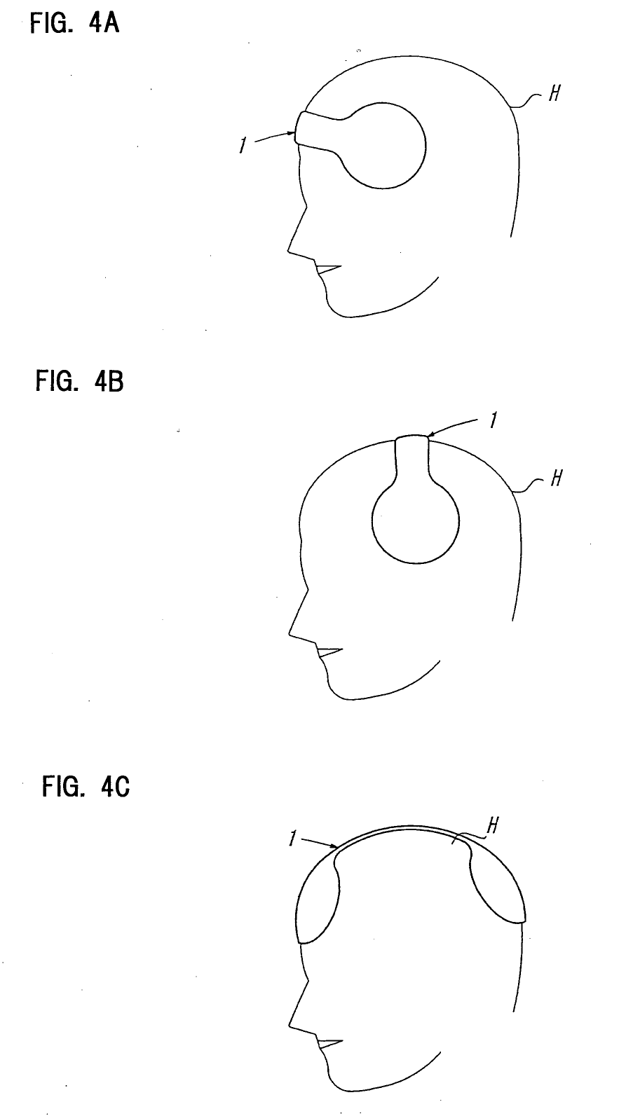

FIG. 1 through FIG. 6(B) shows the first preferred embodiment carrying out the present invention.

As shown in FIG. 1, the first embodiment is comprises of only an extensible, foldable main body 1.

The main body 1 comprises a radiator 11, a cushion member 12, and a covering material 13.

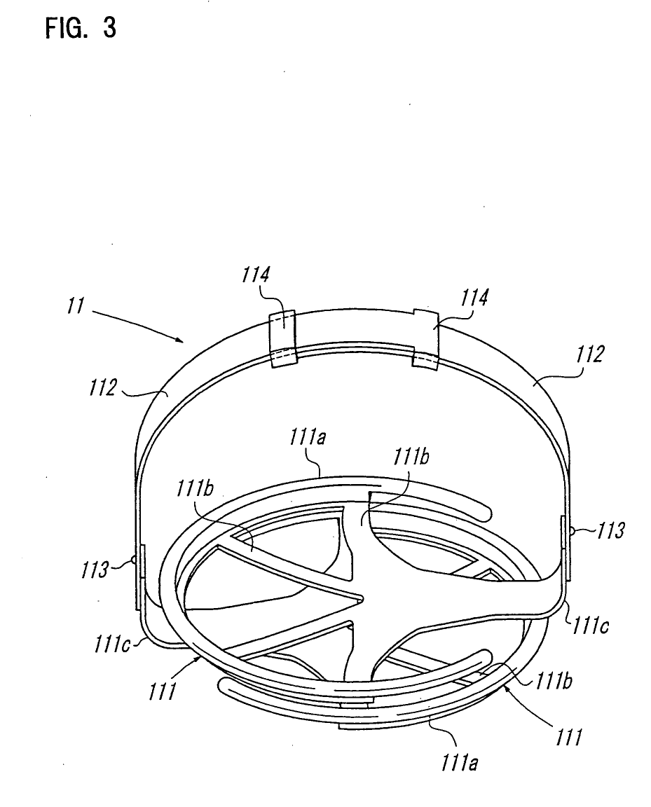

The radiator 11 is formed of elastic thermoplastic synthetic resin (acrylate resin, polypropylene resin or the like) mixed with the far-infrared radiating substance (silica, graphite silica or the like), and comprises cap members 111, a band 112, rotatable joints 113, and a slidable joint 114. The cap member 111 has rims 111a shaped in a partially disconnected ring, arms 111b extending centripetally from the rims, and L-shaped connection pieces 111c extending from the arms 111b. The band 112 comprises two L-shaped band pieces. The rotatable joint 113 has a pin for rotatably connecting the connection piece 111c to one end of the band 112. The slidable joints 114 have insertion frames mounted on the ends of the band 112 so as to permit the opposite ends of the band 112. The radiator 11 is expandable to assume its semicircular shape (cf. FIG. 1) and foldable to be like a handbasket (cf FIG. 3). The far-infrared radiating substance is pulverized into particles having a particle diameter of 0.001 to 0.020 μm and gradually mixed homogeneously into the thermoplastic synthetic resin when plasticating the primary material of the synthetic resin. The mixing ratio of the far-infrared radiating substance is 3 to 10% by weight. When the mixing ratio is less than 3%, a sufficient radiant quantity of the infrared radiation cannot be obtained. When the mixing ratio exceeds 10%, the thermoplastic synthetic resin becomes brittle, so that the strength required for the radiator cannot be obtained. Thus, the ratio by weight of 7 to 8% is the most effective.

The cushion member 12 is made of a foamable resin excellent in affinity for human skin, hygroscopic property and cushioning characteristics and attached mainly to the cap members 111 and the portion in touch with the head H.

The covering material 13 is made of fabrics excellent in affinity for human skin and hygroscopic property and covers the radiator 11 and the cushion member 12.

In this first embodiment, the head orthosis with the spread main body 1 (radiator 11) is put on the head H so as to place the cap members 111 next to the temporal regions of the head H. As a result, the main body 1 is stably retained while coming into steady contact with the temporal regions of the head H by the elasticity of the radiator 11.

Using the first embodiment, the main body 1 can be stably retained with the radiator 11 coming into contact with the temporal regions of the head H, so that the far-infrared rays can be prevented from dispersing over the entire head, thus to apply far-infrared radiation intensively to the head having the nerve tissues. Furthermore, since the radiator 11 of the main body 1 contains homogeneously pulverized far-infrared radiating substance, the far-infrared radiating area of the far-infrared radiating substance is made wide to prevent the interference of the far-infrared radiation. Consequently, the amount of far-infrared radiation releasing from the far-infrared radiating substance can be increased. Moreover, by adjusting the angles of the cap members 111 and the effective length of the band 112 by handling the rotatable joints 113 and slidable joint 114 of the radiator 11, so as to increase wearability of the head orthosis to the temporal regions of the head according to the size and shape of the user's head H.

The head orthosis of the first embodiment can be stored in a compact state by folding the main body 1 (radiator 11) by rotating the connection pieces 111c of the rotatable joints 113 when not in use, as shown in FIG. 3.

FIG. 1 illustrates the state of placing the band 112 of the radiator 11 next to the back of the head.

The inventors of the invention conducted an experiment to ascertain the validity of the present invention in the following manner.

As the far-infrared radiating substance, graphite silica was adopted in the experiment and pulverized into particles having a particle diameter of 0.001 μm. The pulverized far-infrared radiating substance was mixed with Acrylate resin, in a ratio by weight of 7% and formed into the band 112 of the radiator 11, having a width of 25 mm, thickness of 2 mm and length of 28 cm.

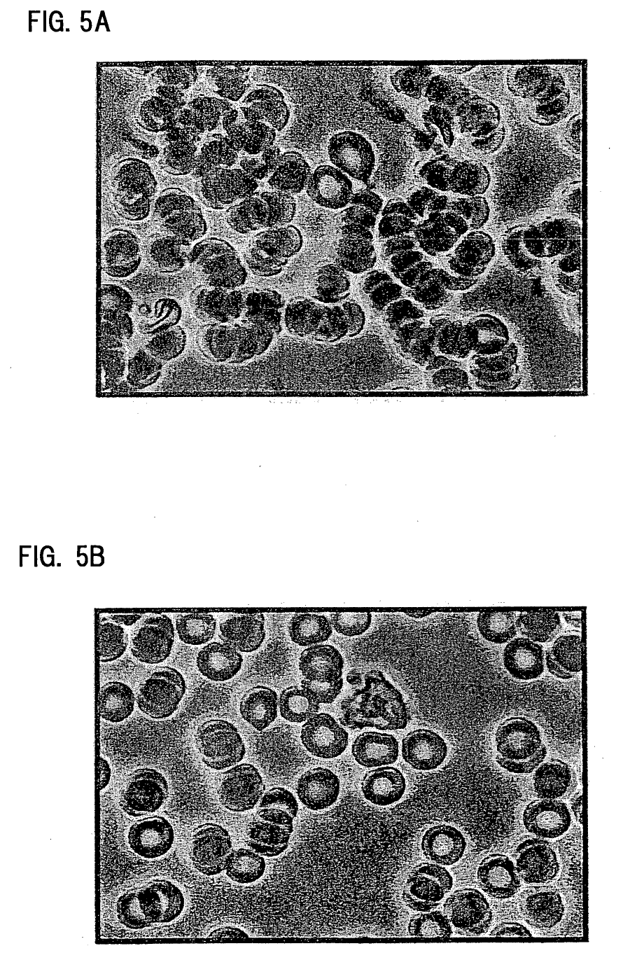

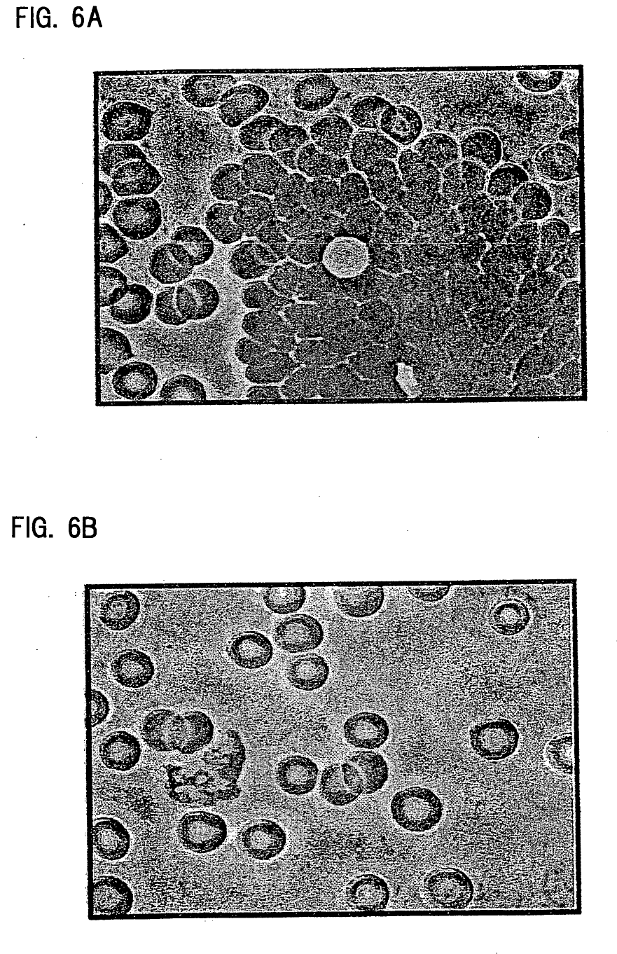

The blood state of an experimental subject (age 42; female) before wearing the head orthosis of the invention is shown in FIG. 5A. The blood state was changed 10 minutes after wearing the head orthosis as shown in FIG. 5B. The blood state of another experimental subject (age 34; female) before wearing the head orthosis as shown in FIG. 6A was changed 10 minutes after wearing to that shown in FIG. 6B.

As a result, obvious blood activation could be founded. Further, the experimental subjects were conscious of alleviation of symptoms such as headache, stiff shoulders, lumbago, and blurred eyesight due to a cataract.

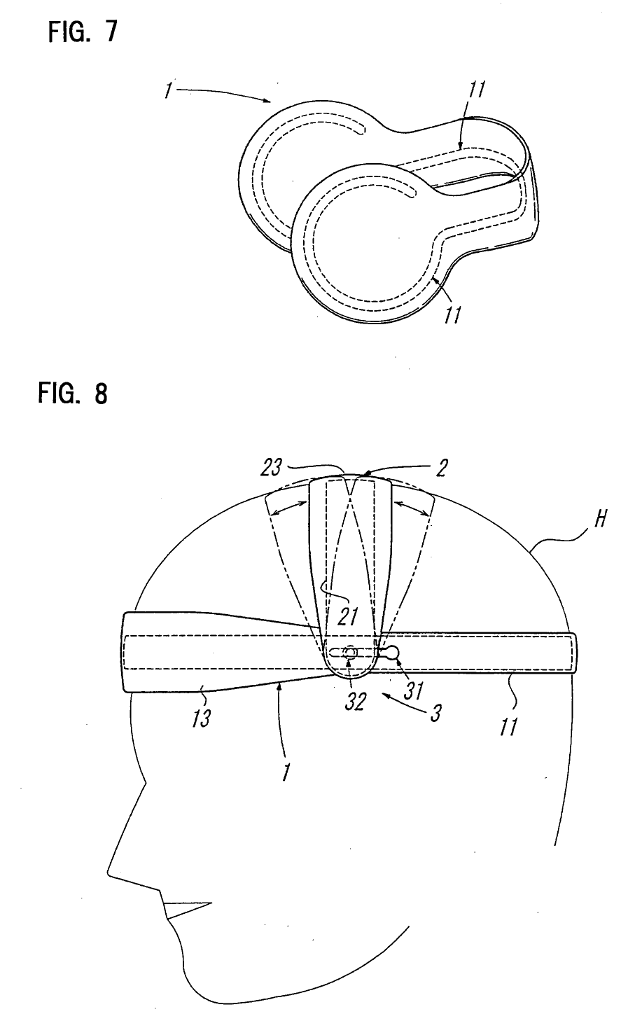

FIG. 7 shows the second preferred embodiment for carrying out the present invention.

The head orthosis in this second embodiment comprises the radiator 11 having the cap members 111 and the band 112 consecutively united in one.

The second embodiment can bring about the fundamentally same function and effect as those of the first embodiment and further has the advantage of capable of simplifying the structure of the radiator 11 and manufacturing the head orthosis of the invention at a moderate price.

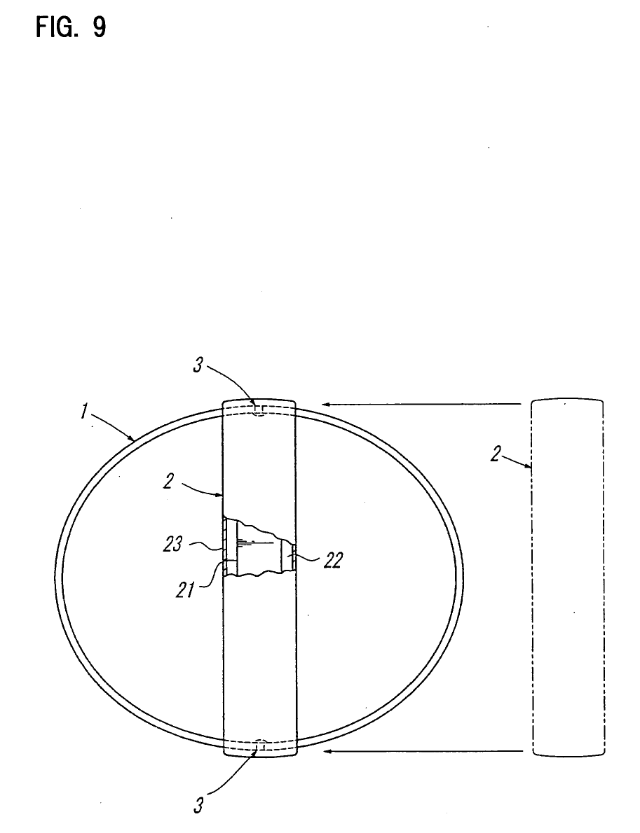

FIG. 8 through FIG. 11 show the third preferred embodiment for carrying out the invention.

The head orthosis in this third embodiment comprises the main body 1, the support member 2, and the connection members 3, as shown in FIG. 8.

The main body 1 comprises the same radiator 11, cushion member 12 and covering material 13 as those in the first embodiment, except the radiator 11 in the third embodiment has a planar shape formed in an elliptical circle so as to bring the sheet-like band in retaining contact with the whole circumference of the temporal region of the head H. Thus, the radiator is made flexible and elastic to some extent in the radial and twisting directions.

The support member 2 comprises a radiator 21, cushion member 22 and covering material 23, which are similar to the radiator 11, cushion member 12 and covering material 13 in the foregoing embodiment. The support member is formed in a substantially semicircle shape so as to come into retaining contact with the parietal region of the head H at the position orthogonal to the main body 1.

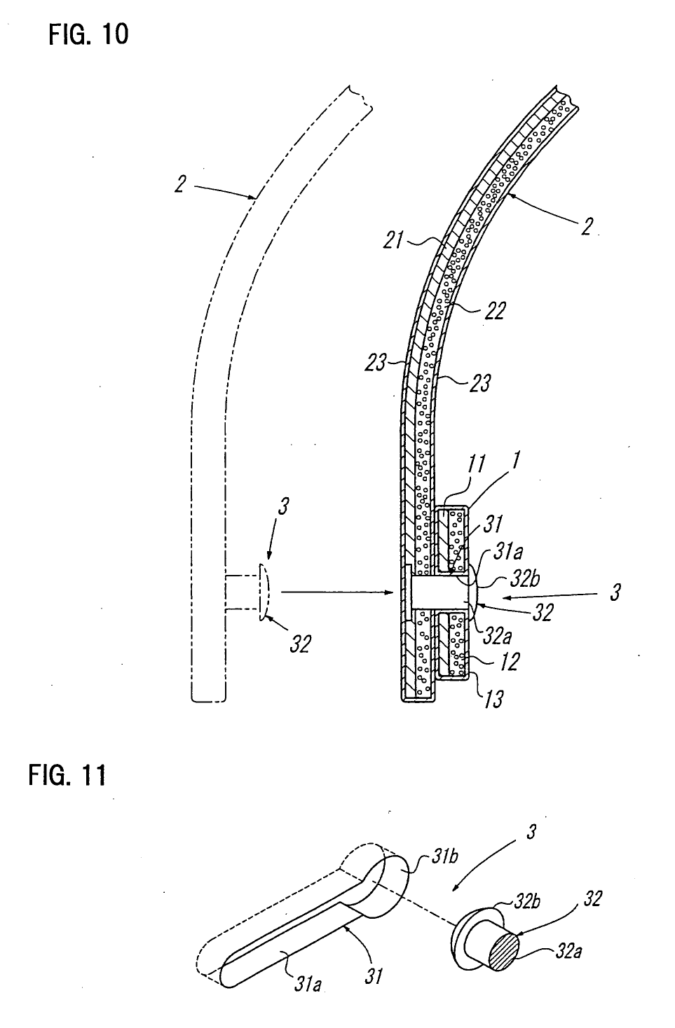

Each connection member 3 comprises a connection catch slot 31 and a connection pin 32 attached to the support member 2, as shown in FIG. 10 in detail.

The connection catch slot 31 is formed of a long hole 31a having a relatively small diameter to pass the covering material 13 therethrough, and a through hole 31b having a relatively large diameter, which is connected with the one end of the long hole 31a. The catch slots each extend in the longitudinal direction (circumferential direction) of the main body 1 and are placed next to the temporal regions on the both sides of the head.

The connection pin 32 has a shank 32a projecting inwardly from each of the end portions of the support member 2 and having a relatively small diameter, and a retaining head 32b formed at the free end of the shank 32a and having a relatively large diameter.

The diameter of the large hole 31a of the connection catch slot 31 is equal to the diameter of the shank 32a of the connection pin 32. The diameter of the through hole 31b of the connection catch slot 31 is equal to the retaining head 32b of the connection pin 32.

According to the third embodiment, the main body 1 and support member 2 can easily be connected and disconnected (disassembling and assembling) by inserting and removing the connection pin 32 into and from the through hole 31b of the connection catch slot 31 in the connection members 3. Thus, the head orthosis can be disassembled and stored in a compact state when not in use.

By sliding the shank 32a of the connection pin 32 of the connection members 3 to the long hole 31a of the connection catch slot 31 after assembling the main body 1 and the support member 2, the main body 1 and the support member 2 can be inextricably connected to prevent the main body 1 from accidentally coming off the support member 2. By making slide adjustment of the shank 32a of the connection pin 32 of the connection member 3 relative to the long hole 31a of the connection catch slot 31 and tilt adjustment of the support member 2 around the shank 32a of the connection pin 32, the support member 2 can be stably fitted in position onto the parietal region of the head according to the size and shape of the user's head H. As a result, when putting the main body 1 and support member 2 on the head H, the main body 1 can be stably retained coming in steady contact with the temporal regions of the head H.

The third embodiment can bring about the fundamentally same function and effect as those of the first embodiment.

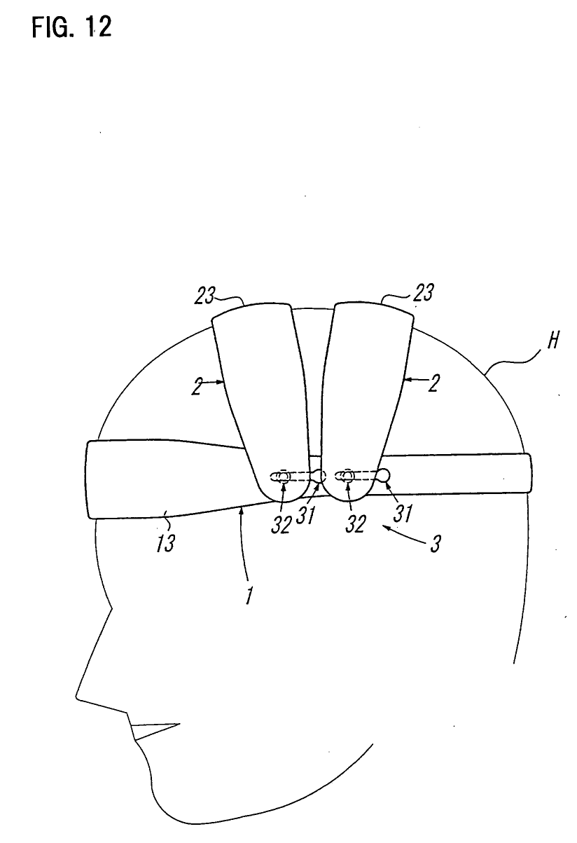

FIG. 12 shows the fourth preferred embodiment for carrying out the invention.

The fourth embodiment has two sets of the support members 2 and connection members 3 found in the third embodiment.

The fourth embodiment can bring about the fundamentally same function and effect as those of the first embodiment and further has the advantage of capable of bringing the support members 2 in stable contact with the parietal region of the head according to the size and shape of the head H by tilting the support members 2 independently.

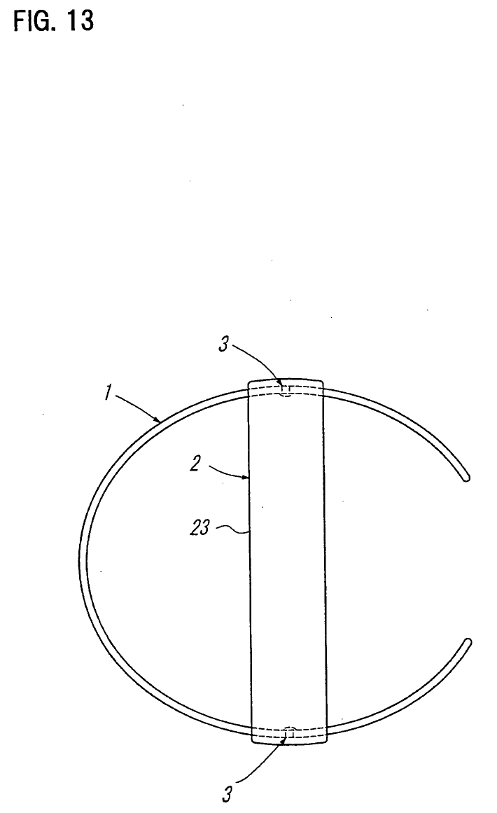

FIG. 13 shows the fifth preferred embodiment for carrying out the invention.

In the fifth embodiment, the main body 1 as found in the third embodiment is partially cut off.

The fifth embodiment can bring about the fundamentally same function and effect as those of the first embodiment and further has the advantage of capable of easily putting the head orthosis on the head irrespective of the size of the head H and hairstyle.

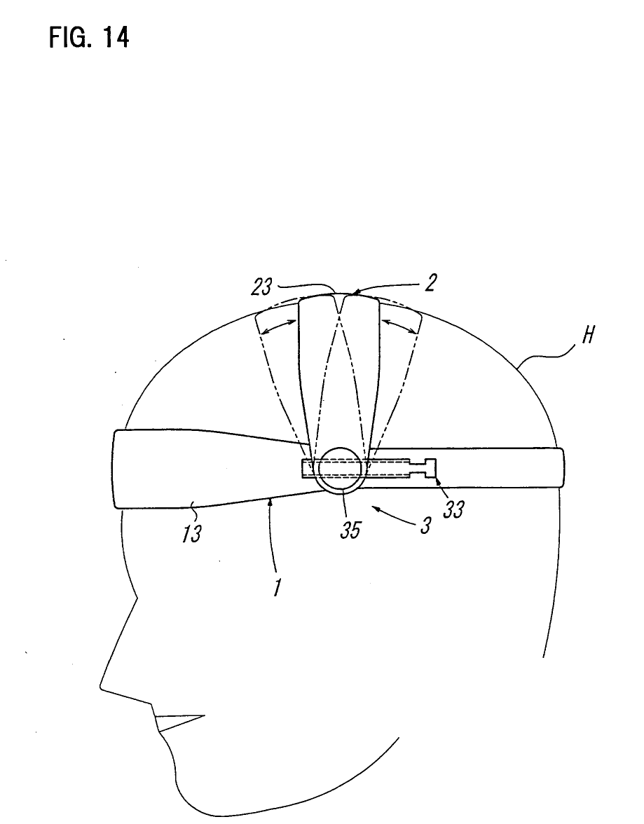

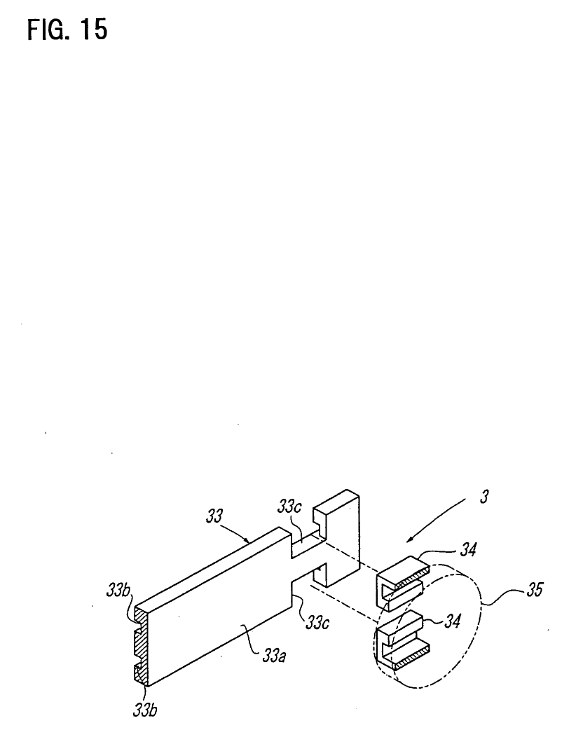

FIG. 14 and FIG. 15 show the sixth preferred embodiment for carrying out the invention.

In this sixth embodiment, the connection member 3 is composed of a rail 33 mounted on the main body 1, a slider 34 attached to the support member 2, and a turning member 35.

The rail 33 is formed in such a manner that rail grooves 33b longitudinally extending in one surface of a short board-like rail body 33a on the side of the main body 1 are connected to cutouts 33c in the end portion of the rail body 33a.

The slider 33 has projections engaged with the rail grooves 33b of the rail 33 retained by the turning member 33. The slider 33 has a width equal to the width between the cutouts 33c of the rail 33.

The turning member 35 is attached to the support member 2 and incorporates a spring and a stopper, so that the support member 2 can be retained stepwise at an arbitrary angle.

The fifth embodiment capable of attaching the rail 33 of the connection member 3 to the outside of the main body 1 eliminates the need for boring a hole in the radiator 11 of the main body 1.

The cutout 33c in the rail 33 of the connection member 3 is applied for connecting and disconnecting the main body 1 to and from the support member 2.

On top of the foregoing embodiments, it is optional to soak the cushion member 12 and covering material 13 of the main body 1 and the cushion member 22 and covering material 23 of the support member 2 with the far-infrared radiating substance including a fixing agent by dipping, applying or printing treatment.

In the alternative, two or more main bodies 1 may be disposed in parallel.

Further, the support member 2 may be made of a material containing no far-infrared radiating substance.

### INDUSTRIAL APPLICABILITY

This invention provides the orthosis worn on the head, but it may be attached to the other parts of the body.

1. A head orthosis comprising a radiator formed of a material containing a pulverized far-infrared radiating substance in a strip shape so as to come into retaining contact with at least a part of the temporal region of the head.

2. The head orthosis claimed in claim 1, wherein the far-infrared radiating substance is silica or graphite silica pulverized with a particle diameter of 0.001 to 0.020 μm.

3. The head orthosis claimed in claim 2, wherein the radiator is formed of thermoplastic synthetic resin mixed with the far-infrared radiating substance in a ratio by weight of 3 to 10%.

4. A head orthosis comprising a radiator formed of a material containing a pulverized far-infrared radiating substance in a strip shape, so as to bring the radiator in retaining contact with at least a part of the temporal region of the head, thereby to elastically hold the radiator onto the temporal region of the head by the elasticity of the material.

5. The head orthosis claimed in claim 4, wherein the far-infrared radiating substance is silica or graphite silica pulverized with a particle diameter of 0.001 to 0.020 μm.

6. The head orthosis claimed in claim 5, wherein the radiator is formed of thermoplastic synthetic resin mixed with the far-infrared radiating substance in a ratio by weight of 3 to 10%.

7. A head orthosis comprising a radiator formed of a material containing a pulverized far-infrared radiating substance in a strip shape, so as to bring the radiator in retaining contact with at least a part of the temporal region of the head, thereby to elastically hold the radiator onto the temporal region by the elasticity of the material and be folded in its detached state from the temporal region.

8. The head orthosis claimed in claim 7, wherein the far-infrared radiating substance is silica or graphite silica pulverized with a particle diameter of 0.001 to 0.020 μm.

9. The head orthosis claimed in claim 8, wherein the radiator is formed of thermoplastic synthetic resin mixed with the far-infrared radiating substance in a ratio by weight of 3 to 10%.

10. A head orthosis comprising a radiator formed of a material containing a pulverized far-infrared radiating substance in a strip shape, said radiator being coupled to a strip-shaped support member in contact with the temporal region of the head.

11. The head orthosis claimed in claim 10, wherein the far-infrared radiating substance is silica or graphite silica pulverized with a particle diameter of 0.001 to 0.020 μm.

12. The head orthosis claimed in claim 11, wherein the radiator is formed of thermoplastic synthetic resin mixed with the far-infrared radiating substance in a ratio by weight of 3 to 10%.

13. A head orthosis comprising a radiator formed of a material containing a pulverized far-infrared radiating substance in a strip shape, said radiator being detachably coupled to a strip-shaped support member in contact with the temporal region of the head, and said radiator and the support member being attachable and detachable with respect to each other.

14. The head orthosis claimed in claim 13, wherein the far-infrared radiating substance is silica or graphite silica pulverized with a particle diameter of 0.001 to 0.020 μm.

15. The head orthosis claimed in claim 14, wherein the radiator is formed of thermoplastic synthetic resin mixed with the far-infrared radiating substance in a ratio by weight of 3 to 10%.

16. A head orthosis comprising a radiator formed of a material containing a pulverized far-infrared radiating substance in a strip shape, said radiator being coupled to a strip-shaped support member coming into contact with the temporal region of the head, wherein the angle between the radiator and the support member is adjustable.

17. The head orthosis claimed in claim 16, wherein the far-infrared radiating substance is silica or graphite silica pulverized with a particle diameter of 0.001 to 0.020 μm.

18. The head orthosis claimed in claim 17, wherein the radiator is formed of thermoplastic synthetic resin mixed with the far-infrared radiating substance in a ratio by weight of 3 to 10%.

19. A head orthosis comprising a radiator formed of a material containing a pulverized far-infrared radiating substance in a strip shape, said radiator being coupled to a strip-shaped support member in contact with the temporal region of the head, wherein the connecting position between the radiator and the support member are adjustable in the longitudinal direction of the radiator.

20. The head orthosis claimed in claim 19, wherein the far-infrared radiating substance is silica or graphite silica pulverized with a particle diameter of 0.001 to 0.020 μm.

21. The head orthosis claimed in claim 20, wherein the radiator is formed of thermoplastic synthetic resin mixed with the far-infrared radiating substance in a ratio by weight of 3 to 10%.
